SEQUENCE LISTING (3 sequences)

SEQ ID NO: 1 - protein, 12 aa, Homo sapiens
AAVLLPVLLAAP

SEQ ID NO: 2 - RNA, 17 nt, mottle virus
  misc_feature (1)..(3): n is a, c, g, or u
nnnbcngucnnnnnnnn

SEQ ID NO: 3 - RNA, 16 nt, mottle virus
  misc_feature (1)..(5): n is a, c, g, or u
nnnnnguannnnnnnn

(19) United States
(12) Patent Application Publication
(10) Pub. No.: US 2005/0026859 A1
(43) Pub. Date: Feb. 3, 2005

(54) Methods and compositions of gene delivery agents for systemic and local therapy

(76) Inventors: John Hilfinger, Ann Arbor, MI (US); Brake Roessler, Ann Arbor, MI (US); Phillip Kish, Ann Arbor, MI (US)

Correspondence Address: GIFFORD, KRASS, GROH, SPRINKLE;ANDERSON & CITKOWSKI, PC, 280 N OLD WOODARD AVE, SUITE 400, BIRMINGHAM, MI, 48009, US

(21) Appl. No.: 10/706,738

(22) Filed: Nov. 12, 2003

Related U.S. Application Data
(60) Provisional application No. 60/425,379, filed on Nov. 12, 2002.

(51) Int. Cl. A61K 48/00; A61K 9/127; C12N 15/88; A61K 31/56

(52) U.S. Cl. 514/44; 424/450; 435/458; 514/171

(57) ABSTRACT

A method is provided for the packaging of a nucleic acid with a chelating agent having a coordinating moiety linked to a central hydrophobic moiety that terminates in a hydrophilic moiety. The complex is well suited for oral and other forms of therapeutic administration of nucleic acids in order to exact systemic and/or localized gene delivery therapy. Intestinal epithelial cells, as well as non-epithelial cells within the gastrointestinal tract and other target cells, are transformed for short or long-term therapies through oral administration, direct injection, or infusive administrations. A nucleic acid conjugating agent particulate composition amenable for administration as a gene therapy composition is provided. The composition is readily adjusted to create a particle having a controlled size and net-zero, -positive, or -negative charge.

### RELATED APPLICATION

This application claims priority of U.S. Provisional Patent Application Ser. No. 60/425,379 filed Nov. 12, 2002, which is incorporated herein by reference.

### FIELD OF THE INVENTION

The present invention relates to gene therapy and more particularly to drug delivery molecules for delivering various nucleic acid compositions to the lumen of the intestine or to localized tissues.

### BACKGROUND OF THE INVENTION

Gene therapy has garnered considerable attention as a method to treat various human diseases by the enhancement of protein production. These include gene replacement or gene augmentation.

The delivery of genetic material into a multi-celled organism has proven more difficult than initially imagined. A variety of techniques have been developed to accomplish in vivo transformation of cells including direct injection of nucleic acid or a particle decorated with nucleic acid directly into cells, recombinant viruses, liposomes and receptor mediated endocytosis.

In attempting to develop lower cost modes of administration that are likely to enhance patient compliance, intestinal gene therapy has been recognized as an attractive situs for in vivo gene therapy owing to the ease of access through oral or rectal routes. However, the intestine routinely degrades large quantities of foreign nucleic acid ingested as part of foodstuffs. DNAses and RNAses in the intestinal tract represent a significant barrier to the entry of intact and functional nucleic acids to intestinal tract cells.

U.S. Pat. No. 6,225,290 represents an effort to deliver bare nucleic acid sequences through laparotomy, oral or suppository administration but is silent as to nucleic acid protection and overcoming the above-stated problems of intestinal administration. It is generally agreed that an oral administration of nucleic acids represents the least expensive and most likely route for the attainment of patient compliance with dosing requirements.

While oral administration is generally recognized as the superior route, little attention has been paid to methodologies and packaging of DNA to preclude intestinal degradation. U.S. Pat. No. 6,500,807 is representative of an attempt to produce a protective coating of carbohydrate around a nucleic acid to facilitate oral administration. It would be advantageous to deliver nucleic acids in a form other than micelles to facilitate conventional pharmaceutical compounding. Thus, there exists a need for gene delivery agents amenable to efficient transfection of target cells capable of inducing systemic and/or local transfection.

### SUMMARY OF THE INVENTION

A method is provided for the packaging of a nucleic acid with a chelating agent having a coordinating moiety linked to a central hydrophobic moiety that terminates in a hydrophilic moiety. The complex is well suited for oral and other forms of therapeutic administration of nucleic acids in order to exact systemic and/or localized gene delivery therapy. Intestinal epithelial cells, as well as non-epithelial cells within the gastrointestinal tract and other target cells, are transformed for short or long-term therapies through oral administration, direct injection, or infusive administrations. In a preferred embodiment, a nucleic acid conjugating agent contains a bile acid linked with a polycationic peptide.

A nucleic acid conjugating agent particulate composition amenable for administration as a gene therapy composition is provided. The composition is readily adjusted to create a particle having a controlled size and net-zero, -positive, or -negative charge. The resulting composition is optionally provided in a pharmaceutically acceptable carrier for administration to achieve gene therapy, elicit an immunological response, label transfected cells, or deliver associated pharmaceutically active agents therewith.

### BRIEF DESCRIPTION OF THE DRAWINGS

FIG. 1 is transmission electron microscopy (TEM) of a bile acid conjugate (BAC)/plasmid DNA (pDNA) complex;

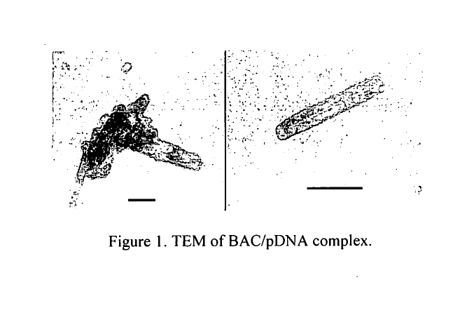

FIGS. 2a-2d are relative percent particle size bar graphs of: (FIG. 2a) pDNA; (FIG. 2b) pDNA and 50 μg/ml BAC initially upon mixing and corresponding to a negatively charged complex; (FIG. 2c) pDNA and 200 μg/ml BAC initially upon mixing and corresponding to a neutrally charged complex; and (FIG. 2d) the complex depicted in FIG. 2c after two hours of mixing;

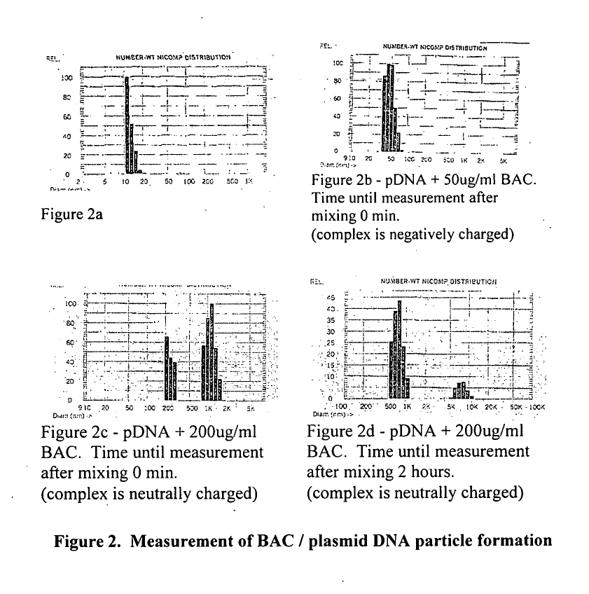

FIGS. 3a-3c are relative percent particle size bar graphs of inventive BAC-pDNA particles having a relative ratio of BAC:pDNA of 80,699:1 in: (FIG. 3a) water; (FIG. 3b) simulated intestinal fluid; and (FIG. 3c) simulated gastric fluid;

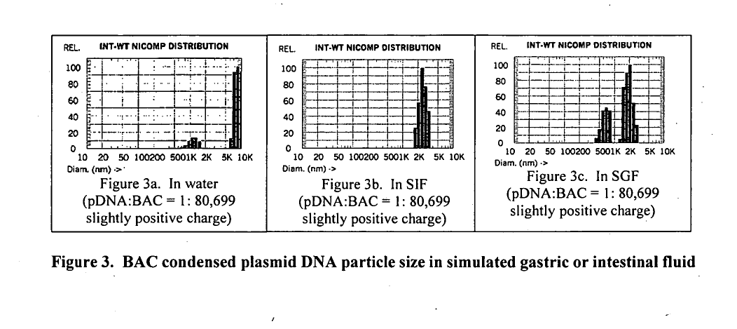

FIG. 4 is a plot of the zeta potential measured as a function of conjugating agent:DNA mol ratio where (●) indicates addition of further conjugating agent after particle formation and (▴) indicates the addition of additional nucleic acid after particle formation;

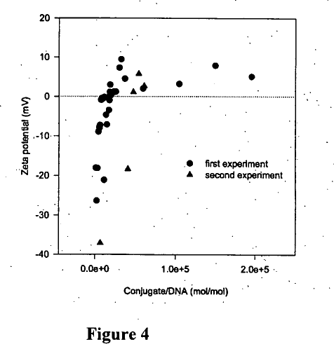

FIG. 5 is an electrophoretic gel showing the relative mobility of nucleic acid, alone and in combination with at least one of conjugating agent, polyaspartic acid and DNAse I;

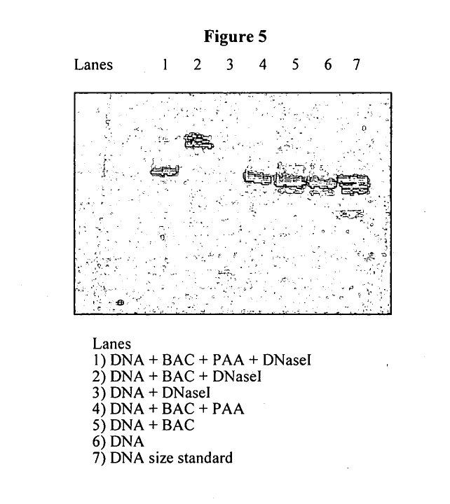

FIG. 6 is a bar graph showing in vitro transfection of Hela cells with BAC-luciferase pDNA at various conjugating agent:pDNA ratios; and

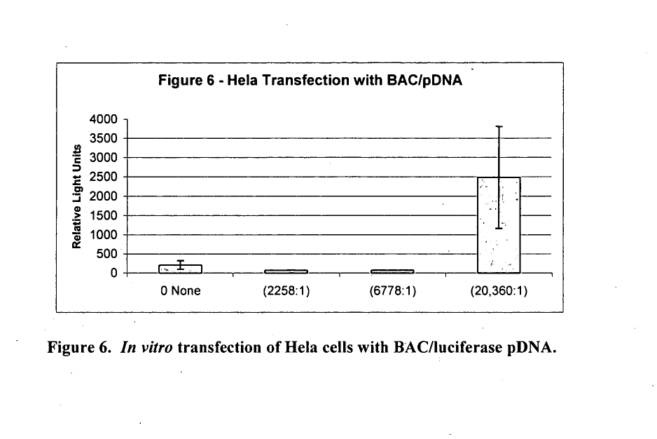

FIG. 7 is a plot of inventive complex single pass absorption for neutrally charged and positively charged radiolabeled BAC-pDNA as a function of time where filled symbols indicate jejunal absorption and open symbols indicate ileal absorption.

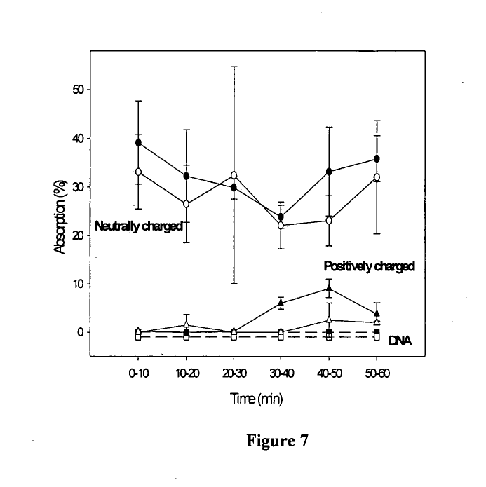

### DETAILED DESCRIPTION OF THE PREFERRED EMBODIMENTS

The present invention has utility as a treatment for a variety of disease conditions or deficiencies. These conditions and deficiencies illustratively include: enzyme deficiency, erythropoietin, catalase, endotoxic shock/sepsis, adenosine deaminase for treatment of severe combined immunodeficiency, lipid-binding protein (LBP), purine nucleotide phosphorylase, galactosidase, beta-glucuronidase, antioxidants for cancer, therapy anemia, superoxide dismutase, cancer, growth factors for use in wound healing, induction of red blood cell formation and the like, α-interferon, β-interferon, epidermal growth factor, granulocyte colony stimulating factor (G-CSF), alpha-IL1, gamma-interferon, phenylalanine ammonia lyase, transforming growth factor, arginase, erythropoietin, L-asparaginase, thrombopoietin, uricase, insulin-like growth factor-1, insulin, human growth hormone, monoclonal antibodies, tissue necrosis factor, cardiovascular disease, diabetes, tissue plasminogen activator, urokinase (native or chimeric), glucagon, α₁-antitrypsin, insulinotrophic hormone, clotting disorders, antithrombin-III, other proteases or protease inhibitors, clotting factor VIII, apolipoproteins (particularly B-48), circulating scavenger receptor, APO A1 which converts low-density lipoproteins to high-density lipoproteins, gastrointestinal and pancreatic deficiencies, obesity and feeding, pepsin (for esophageal reflux), Ob gene product, cholecystokinin (CCK), trypsin, chymotrypsin, bone diseases, elastase, carboxypeptidase, calcitonin, lactase (for lactose deficiency), PTH-like hormone, sucrase, intrinsic factor (pernicious anemia), myasthenia gravis (acetylcholine receptors), Graves' disease (thyroid-stimulating hormone receptor), organ-specific autoimmune diseases (target of antibody in parentheses), thyroiditis (thyroid, peroxidase), insulin-resistant diabetes with acanthosis nigricans or with ataxia telangiectasia (insulin receptor), allergic rhinitis, asthma (β₂-adrenergic receptors), juvenile insulin-dependent diabetes (insulin, GAD65), pernicious anemia (gastric parietal cells, vitamin B₁₂ binding site of intrinsic factor), Addison's disease (adrenal cells), idiopathic hypoparathyroidism (parathyroid cells), spontaneous infertility (sperm), premature ovarian failure (interstitial cells, corpus luteum cells), pemphigus (intercellular substance of skin and mucosa), bullous pemphigoid (basement membrane zone of skin and mucosa), primary biliary cirrhosis (mitochondria), autoimmune hemolytic anemia (erythrocytes), idiopathic thrombocytopenic purpura (platelet), idiopathic neutropenia (neutrophils), vitiligo (melanocytes), osteosclerosis and Meniere's disease (type II collagen), chronic active hepatitis. (nuclei of hepatocytes), systemic autoimmune diseases (defect/organ affected in parentheses), Goodpasture's syndrome (basement membranes), rheumatoid arthritis (γ-globulin, EBV-related antigens, collagen types II and III), Sjogren's syndrome (γ-globulin, SS-A (Ro), SS-B (La), systemic lupus erythematosus (nuclei, double-stranded DNA, single-stranded DNA, Sm ribonucleoprotein, lymphocytes, erythrocytes, neurons, gamma-globulin), scleroderm (nuclei, Scl-70, SS-A (Ro), SS-B (La), centromere, polymyositis (nuclei, Jo-1, PL-7, histadyl-tRNA synthetase, threonyl-tRNA synthetase, PM-1, Mi-2), rheumatic fever (myocardium heart valves), and choroid plexus.

The invention involves methods and products for oral, parenteral, mucosal, and infusion delivery of nucleic acid for both systemic and localized therapy. Depending on the nature of the nucleic acid sequence, these contain, in non-covalently bound form, one or more substances having an affinity for nucleic acid, which are capable of increasing the efficiency of absorption of the complexes into the cells. Cells of a mammalian subject, either intestinal epithelia after oral delivery, or cells in other organs after other forms of inventive delivery, are altered to operatively incorporate a gene which expresses a protein, which is secreted directly into the organ and/or blood stream to provide a therapeutic effect. The use of naked nucleic acid protected by complexation with adsorption and/or internalization factors avoids the complications associated with use of viral vectors to accomplish gene therapy. An inventive complex is delivered via the intestinal lumen in a variety of ways, including through timed-release capsules, such as those detailed in U.S. Pat. No. 4,976,949, thereby obtaining a simple, noninvasive method of gene delivery. These complexes also optionally are delivered to other organs of the body in a variety of ways, including direct injection or infusion.

As used herein, a “gene” is defined to be an isolated nucleic acid molecule of greater than twenty nucleotides. A gene operative herein is recognized to be one that illustratively replaces or supplements a desired function, or achieves a desired effect such as the inhibition of tumor growth or induction of an immune response to the gene itself or a polypeptide transcribed therefrom. It is appreciated that a nucleic acid molecule according to the present invention illustratively includes plasmids, vectors, external guide sequences for RNAase, ribozymes, DNA, RNA, and miRNA. Antisense nucleic acids sequences are also administered according to the present invention. A gene is generally under the control of an appropriate promoter, which may be inducible, repressible, or constitutive. Promoters can be general promoters, yielding expression in a variety of mammalian cells, or cell specific, or even nuclear versus cytoplasmic specific. Viral promoters such as CMV are also operative herein. These are known to those skilled in the art and can be constructed using standard molecular biology protocols.

In a preferred embodiment administration is oral and targeted to transfect intestinal epithelial cells.

As used herein, a “subject” includes humans, non-human primates, horses, goats, cows, sheep, pigs, dogs, cats, and rodents. The methods and compounds of the present invention are administered in therapeutically effective amounts.

As used herein, a “therapeutically effective amount” is defined to include an amount necessary to delay the onset of, inhibit the progress of, relieve the symptoms of, or reverse a condition being treated; induce an immune response to the delivered gene or a polypeptide encoded thereby or regulate the expression of an existing cellular product. The therapeutically effective amount is one that is less than that that produces medically unacceptable side effects. It is appreciated that a therapeutically effective amount varies with a number of factors illustratively including subject age, condition, sex and the nature of the condition being treated. It is further appreciated that determining a therapeutically effective dose is within the knowledge of one of ordinary skill in the art.

As used herein, however, the term “peptide” is intended to include mimetics and is used synonymously with polypeptide. The term “amino acid” is intended to include D-form amino acids and modified amino acids.

The compounds of the present invention are administered to a subject at dosage levels in the range of about 0.000002 mg/m² to about 4 mg/m² of conjugating agent combined with about 0.2 mg/m² to about 4 mg/m² of nucleic acid per day. For a normal human adult having a body weight of about 70 kg, a dosage in the range of about 0.005-10 mg/kg/day conjugating agent combined with about 5×10⁻⁶-10 mg/kg/day nucleic acid is preferable. The general ratio of the amount of conjugating agent to the nucleic acid ranges from about 50:1-500,000:1 in the composition which is administered to a subject.

After oral delivery the transformed intestinal epithelial cells provide short or long term therapies for diseases associated with a deficiency in a particular protein or which are amenable to treatment or palliation by over expression of a protein including metabolic disorders, endocrine disorders, circulatory disorders, coagulation disorders, cancer, and gastrointestinal disease.

An inventive conjugating agent has the general formula:
A-R₁-Q-Y-Z
where A-R₁ is a cholesterol derivative; a C₈-C₂₄ alkyl; C₈-C₂₄ heteroatom substituted alkyl wherein the heteroatom is O, N or S; where A is a hydrophilic moiety A that illustratively includes C₀-C₄ alkyl-hydroxy, -substituted amino, -quaternary amino, -sulfonate, -phosphonate, and -carboxylate; and targeting ligand; where the targeting ligand includes amino acids, hormones, antibodies, cell adhesion molecules, folate, polypeptides, vitamins, saccharides, transferring, drugs, and neurotransmitters; where Q is sulfur, a secondary amine, or oxygen; where Y is a linker peptide having a negative, neutral, or positive charge; and where Z is a polyionic peptide. Specific examples of inventive cholesterol derivatives illustratively include cholestanol, coprostanol, cholic acid, glycocholic acid, chenodeoxycholic acid, desoxycholic acid, glycochenodeoxycholic acid, taurocholic acid, and taurochenodeoxycholic acid. Specific examples of C₈-C₂₄ alkyls are 13-hydroxyl tridecanoic acid; 1,12 dodecane diol; and 1,12 dodecanediame.

A peptide linker sequence Y is preferably employed to separate A-R₁-Q and the polyionic peptide sequence Z that interacts with the nucleic acid by a distance sufficient to ensure that the cholesterol derivative is stearically accessible and that the polyionic peptide Z folds into its secondary and tertiary structures. Such a peptide linker sequence Y is incorporated into an inventive compound using standard techniques well known in the art. Suitable peptide linker sequences Y are chosen based on the following factors: (1) their ability to adopt a flexible extended conformation; (2) their inability to adopt a secondary structure that could interact with functional epitopes of the inventive compound; and (3) the lack of hydrophobic or charged residues that might react with the polyionic peptide functional epitopes. Preferred peptide linker sequences contain Gly, Asn and Ser residues. Other near-neutral amino acids, such as Thr and Ala, also are operative in the linker sequence. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. Nos. 4,935,233 and 4,751,180. The linker sequence may be from 0 to about 50 amino acids in length. A peptide linker sequence Y is not required when the polyionic peptide Z has non-essential N-terminal amino acid regions that can be used to separate the functional domains and prevent steric interference.

A polyionic peptide Z according to the present invention is generally a highly charged polypeptide or protein, having an isoelectric point of between about 3 to about 12. The polyionic peptide Z is generally soluble in salt-free, aqueous solution. Illustrative polyionic peptides include, but are not limited to, one or more of the following: albumin, such as from egg or animal, e.g. bovine, serum; derivatized collagen polypeptides, such as cationic collagen polypeptides; elastin; globulin polypeptide, such as myoglobin; synthetic polypeptides rich in glutamic acid, aspartic acid, lysine or arginine residues, such as polyaspartic acid; and derivatives of such proteinaceous or other materials, such as keratin. A particularly preferred polyionic peptide Z is polyaspartic acid. It is appreciated that in the formation of synthetic polyionic peptides that the inclusion of both cationic and anionic amino acid residues create a complex charge on an inventive compound under pH conditions that vary from the isoelectric point.

Proteins usefully expressed according to the administration of the present invention illustratively include proteases, pituitary hormones, protease inhibitors, growth factors, cytokines, somatomedians, chemokines, immunoglobulins, gonadotrophins, interleukins, chemotactins, interferons, and lipid-binding proteins, specific examples of which illustratively include insulin, interferon-α2B, human growth hormone (hGH), transforming growth factor (TGF), erythropoietin (EPO), ciliary neurite transforming factor (CNTF), clotting factor VIII, insulin-like growth factor-1 (IGF-1), bovine growth hormone (BGH), granulocyte macrophage colony stimulating factor (GM-CSF), platelet derived growth factor (PDGF), interferon-α2A, clotting factor VIII, brain-derived neurite factor (BDNF), thrombopoietin (TPO), insulintropin, tissue plasminogen activator (tPA), IL-1, IL-2, urokinase, IL-1 RA, streptokinase, superoxide dismutase (SOD), adenosine deamidase, catalase, calcitonin, arginase, fibroblast growth factor (FGF) (acidic or basic), neurite growth factor (NGF), phenylalanine ammonia lyase, granulocyte colony stimulating γ-interferon factor (G-CSF), L-asparaginase, pepsin, uricase, trypsin, chymotrypsin, elastase, carboxypeptidase, lactase, sucrase, intrinsic factor parathyroid hormone (PTH)-like hormone, calcitonin, Ob gene product, cholecystokinin (CCK), glucagon, glucagon-like-peptide I (GLP-1), and insulinotrophic hormone.

The conjugate agent A-R₁-Q-Y-Z is preferably a bile acid conjugated with a polycationic peptide linked to the bile acid steroid backbone. The bile acid moiety acts to target the conjugate to bile acid transporters in the lumen of the intestine and assist in the cellular internalization of the complex. Short polycation peptides rich in arginine or lysine, such as a six amino acid residue or longer chain, provide multiple functions illustratively including: a) having an affinity for nucleic acid, b) act condensing agent, c) protect the nucleic acid from nuclease activity and d) assist in cellular internalization of the complex.

8. A method for treating a disease condition or deficiency through gene delivery to target cells of a subject comprising the step of administering a conjugating agent-nucleic acid complex where the conjugating agent comprises A-R₁-Q-Z, where A-R₁ is a cholesterol derivative; a C₈-C₂₄ alkyl; C₈-C₂₄ heteroatom substituted alkyl wherein the heteroatom is O, N, or S; or a bile acid; Q is a sulfur, a secondary amine or oxygen having a nonessential N-terminal amino acid region; and Z is a polyionic peptide.

9. The method of claim 8, wherein said administration is oral.

10. The method of claim 8, wherein nucleic acid of said complex is expressed as a protein in said target cells.

11. The method of claim 10 wherein said protein is secreted from said target cells.

12. The method of claim 10 wherein said protein is of a class selected from the group consisting of: proteases, pituitary hormones, protease inhibitors, growth factors, cytokines, somatomedians, chemokines, immunoglobulins, gonadotrophins, interleukins, chemotactins, interferons, and lipid-binding proteins.

13. The method of claim 8 wherein said nucleic acid of said complex is selected from the group consisting of: DNA, RNA, mRNA, miRNA, ribozyme, RNAse, and antisense sequences.

14. The method of claim 8 wherein said complex is administered as part of a pharmaceutical composition.

15. The method of claim 14 wherein said pharmaceutical composition comprises an active therapeutic compound.

16. The method of claim 15 wherein said therapeutic agent is selected from the group consisting of: an antibiotic, a gamma or beta radiation emitting species, an anti-inflammatory, an antitumoral, an antiviral, an antibody, a hormone, an enzyme, antigenic peptide and antigenic protein.

17. The method of claim 8 wherein A-R₁ is a cholesterol derivative.

18. The method of claim 17 wherein said A is a hydrophilic moiety.

19. The method of claim 8, wherein said target cells are gastrointestinal cells.

20. A gene delivery composition comprising a conjugating agent-nucleic acid complex having the formula:
A-R₁-Q-Y-Z
where A-R₁ is a cholesterol derivative; a C₈-C₂₄ alkyl; C₈-C₂₄ heteroatom substituted alkyl wherein the heteroatom is O, N or S; where A is a hydrophilic moiety A that illustratively includes C₀-C₄ alkyl-hydroxy, -substituted amino, -quaternary amino, -sulfonate, -phosphonate, and -carboxylate and a target ligand; where Q is sulfur, nitrogen, or oxygen; where Y is a linker peptide having a negative, neutral, or positive charge; and where Z is a polyionic peptide.

21. The composition of claim 20 wherein said cholesterol derivative is selected from the group consisting of: cholestanol, coprostanol, cholic acid, glycocholic acid, chenodeoxycholic acid, desoxycholic acid, glycochenodeoxycholic acid, taurocholic acid, and taurochenodeoxycholic acid.

22. The composition of claim 20 wherein said cholesterol derivative is a cholic acid or a deoxycholic acid.

23. The composition of claim 20 wherein said A derivative is hydroxyl.

24. The composition of claim 20 wherein said Q derivative is oxygen.

25. The composition of claim 20 wherein Y and Z together yield a net neutral charge.

26. The composition of claim 20 wherein Z is polycationic.

27. The composition of claim 26 wherein Z contains at least six residues.

28. Use of a bile acid salt as a conjugating agent to administer nucleic acid to a subject.

29. The use of claim 28 wherein administration is oral.

30. A commercial package comprising a composition of Formula I according to claim 8 as an active ingredient together with instructions for the use thereof as a gene delivery agent to a subject.
